(19) United States
(12) Patent Application Publication
(10) Pub. No.: US 2018/0064408 A1
(43) Pub. Date: Mar. 8, 2018

(54) RADIATION IMAGING APPARATUS, RADIATION IMAGING SYSTEM, RADIATION IMAGING METHOD, AND COMPUTER-READABLE MEDIUM

(71) Applicant: CANON KABUSHIKI KAISHA, Tokyo (JP)

(72) Inventors: Tetsuo Shimada, Hachioji-shi (JP); Osamu Tsujii, Kawasaki-shi (JP)

(21) Appl. No.: 15/686,548

(22) Filed: Aug. 25, 2017

(30) Foreign Application Priority Data
Sep. 6, 2016 (JP) 2016-173301

(51) Int. Cl. A61B 6/00; A61B 6/03

(52) U.S. Cl. CPC A61B 6/4417; A61B 6/502; A61B 6/4435; A61B 6/5235; A61B 6/032; A61B 6/463

(57) ABSTRACT

Provided is a radiation imaging apparatus including: an image acquiring unit configured to acquire a first radiation image, which is photographed by irradiating an object with radiation under a state in which a subject is not in an imaging range, and a second radiation image, which is photographed by radiating radiation under a state in which the subject and the object are in the imaging range; and an image processing unit configured to perform correction for deleting image information on the object from the second radiation image by using the first radiation image.

### BACKGROUND OF THE INVENTION

### Field of the Invention

The present invention relates to a radiation imaging apparatus configured to perform imaging using radiation, a radiation imaging system, a radiation imaging method, and a computer-readable medium.

### Description of the Related Art

As a breast imaging apparatus, there is a computed tomography (CT) apparatus configured to photograph an image of the breast of a subject by rotating a radiation generation unit configured to generate radiation and a radiation detection unit configured to detect radiation by a rotation unit. For example, there is disclosed a CT apparatus configured such that an X-ray generation unit and an X-ray detection unit arranged facing each other are rotated about an opening for inserting the breast (e.g., refer to Japanese Patent Application Laid-Open No. 2010-068929).

For a mammography CT apparatus in which imaging is performed in a standing position or a semi-standing position, there has been proposed a method of performing the imaging by placing the breast in a breast cup fixed to the apparatus in order to prevent the breast from hanging down during imaging. In the case of a three-dimensional display in which the breast and the breast cup are displayed during reading of the image, reading may be impaired because the shadow of the breast cup is displayed surrounding the breast.

### SUMMARY OF THE INVENTION

A radiation imaging apparatus according to one embodiment of the present invention includes: an image acquiring unit configured to acquire a first radiation image, which is photographed by irradiating an object with radiation under a state in which a subject is not in an imaging range, and a second radiation image, which is photographed by radiating radiation under a state in which the subject and the object are in the imaging range; and an image processing unit configured to perform correction for deleting image information on the object from the second radiation image by using the first radiation image.

Further features of the present invention will become apparent from the following description of exemplary embodiments with reference to the attached drawings.

### BRIEF DESCRIPTION OF THE DRAWINGS

FIG. 1 is a view for illustrating an appearance of a radiation imaging system according to an embodiment of the present invention.

FIG. 2 is a view for illustrating the appearance of the radiation imaging system as viewed from a CT imaging side.

FIG. 3 is a cross-sectional view for illustrating the radiation imaging system according to the embodiment of the present invention.

FIG. 4 is a view for illustrating the appearance of the radiation imaging system as viewed from a mammographic imaging side.

FIG. 5 is a diagram for illustrating a configuration of the radiation imaging system according to the embodiment of the present invention.

FIG. 6 is a view for illustrating a rotation mode in which a radiation generation unit and a radiation detection unit of the embodiment of the present invention have been rotated.

FIG. 7 is a diagram for illustrating an example of a process configuration for mammographic image correction.

FIG. 8 is a diagram for illustrating an example of a process configuration for CT image correction in which an object and a subject are included.

FIG. 9 is a diagram for illustrating an example of a process configuration for CT image correction including the subject under a state in which the object has been deleted.

FIG. 10 is a diagram for illustrating an example of a process configuration for CT image correction of the object under a state in which there is no subject.

FIG. 11 is a diagram for illustrating an example of an image including the subject and the object.

FIG. 12 is a diagram for illustrating an example of an image including the subject under a state in which the object has been deleted.

FIG. 13 is a diagram for illustrating an example of an image of the object under a state in which there is no subject.

### DESCRIPTION OF THE EMBODIMENTS

Preferred embodiments of the present invention will now be described in detail in accordance with the accompanying drawings.

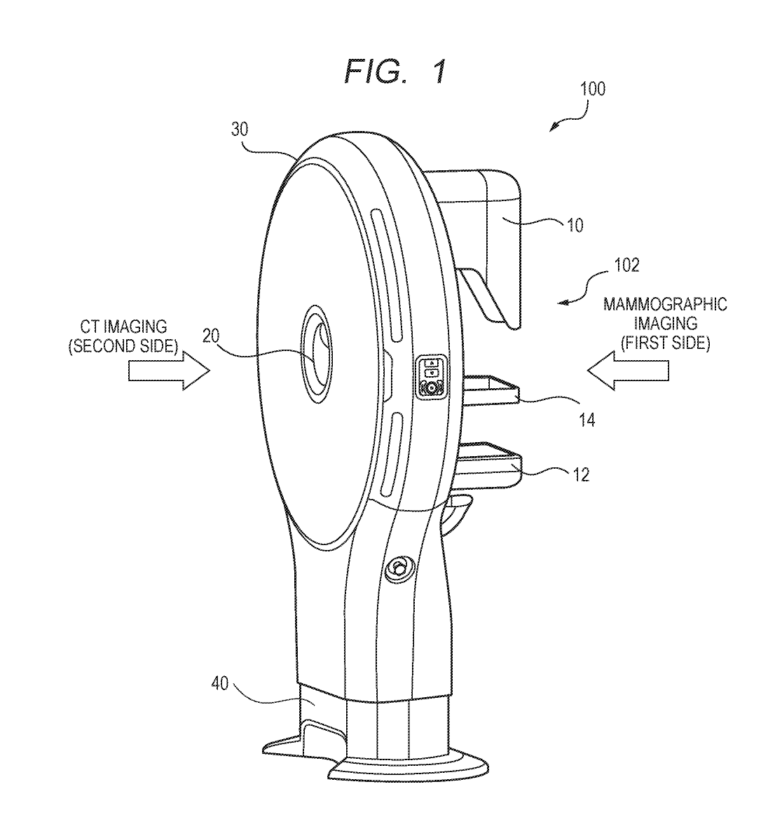

FIG. 1 is a view for illustrating an appearance of a radiation imaging system (breast imaging system) 100 including a radiation imaging apparatus (breast imaging apparatus). The breast imaging system 100 is capable of performing mammographic imaging and CT imaging. When mammographic imaging and CT imaging are performed, a subject is in a standing position, which is a state in which the subject is standing with both feet placed on a floor surface. More specifically, the breast imaging system 100 is a standing-type breast imaging apparatus.

The breast imaging system 100 includes a radiation generation unit 10 configured to generate radiation, and a radiation detection unit 12 configured to detect radiation radiated from the radiation generation unit 10. The radiation generation unit 10 is rotatable about the subject, and is configured to generate radiation. The radiation detection unit 12 is arranged facing the radiation generation unit 10, is rotatable together with the radiation generation unit 10, and is configured to detect radiation. The radiation generation unit 10 and the radiation detection unit 12 can be rotated under a state of facing each other. An imaging unit 102 mainly includes the radiation generation unit 10 and the radiation detection unit 12.

In the breast imaging system 100, imaging is performed from a first side under a state in which a site (i.e., breast) to be imaged of the subject has been sandwiched between a compression plate 14 and the radiation detection unit 12. More specifically, the breast imaging system 100 has a mammographic imaging mode. Further, in the breast imaging system 100, imaging is performed from a second side, which is opposite from the first side, by inserting the site (i.e., breast) to be imaged of the subject between the radiation generation unit 10 and the radiation detection unit 12, placing the site (i.e., breast) to be imaged in a breast cup, and rotating the radiation generation unit 10 and the radiation detection unit 12 under this state. More specifically, the breast imaging system 100 has a CT imaging mode.

The breast imaging system 100 includes a gantry 30 configured to rotatably support the radiation generation unit 10 and the radiation detection unit 12, and a support leg 40 configured to support the gantry 30 on the floor surface. In other words, the gantry 30 is configured to rotatably support the imaging unit 102.

When mammographic imaging is performed, the imaging is performed from the first side (i.e., right side in FIG. 1) of the breast imaging system 100 under a state in which the site (i.e., breast) to be imaged of the subject has been sandwiched between the compression plate 14 and the radiation detection unit 12.

The compression plate 14 is a transparent material through which radiation passes. Specifically, the breast of the subject can be sandwiched between the compression plate 14 and the radiation detection unit 12 by moving the compression plate 14 up or down. Radiation is generated by the radiation generation unit 10 under a state in which the breast of the subject has been sandwiched between the compression plate 14 and the radiation detection unit 12. The breast of the subject can be imaged by using the radiation detection unit 12 to detect the radiation that has passed though the breast of the subject. The breast imaging system 100 can generate a mammographic image based on the radiation data acquired by the imaging.

When CT imaging is performed, the site (i.e., breast) to be imaged of the subject is inserted between the radiation generation unit 10 and the radiation detection unit 12 from the second side (i.e., left side in FIG. 1), which is opposite from the first side, of the breast imaging system 100. Imaging is then performed under this state by rotating the radiation generation unit 10 and the radiation detection unit 12 by a rotating frame 38.

An opening 20 for inserting the breast of the subject is arranged in the gantry 30 of the breast imaging system 100. Imaging is performed by rotating the radiation generation unit 10 and the radiation detection unit 12 by the rotating frame 38 under a state in which the breast of the subject has been inserted through the opening 20 and placed in the breast cup. During the period in which the radiation generation unit 10 and the radiation detection unit 12 are being rotated by the rotating frame 38, radiation is generated by the radiation generation unit 10. The radiation detection unit 12 is capable of imaging the breast of the subject by detecting the radiation that has passed through the breast of the subject. The breast imaging system 100 can generate a CT image by reconstructing the radiation data acquired by the imaging.

The first side in the breast imaging system 100 is the mammographic imaging side, and the second side in the breast imaging system 100 is the CT imaging side. A line horizontally connecting the first side (mammographic imaging side) and the second side (CT imaging side) is substantially parallel to the axis of rotation of the rotating frame 38. The line horizontally connecting the first side (mammographic imaging side) and the second side (CT imaging side) is orthogonal to a plane of the substantially plate-shaped gantry 30, or to a plane of a front cover 26.

The first side (mammographic imaging side) and the second side (CT imaging side) in the breast imaging system 100 are areas divided by the substantially plate-shaped gantry 30, the front cover 26, and the imaging unit 102 of the breast imaging system 100.

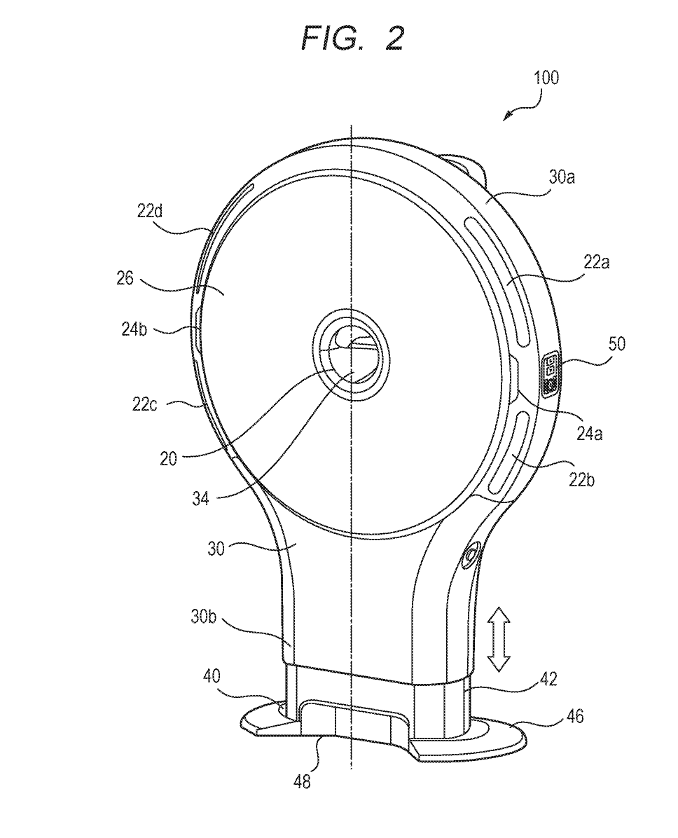

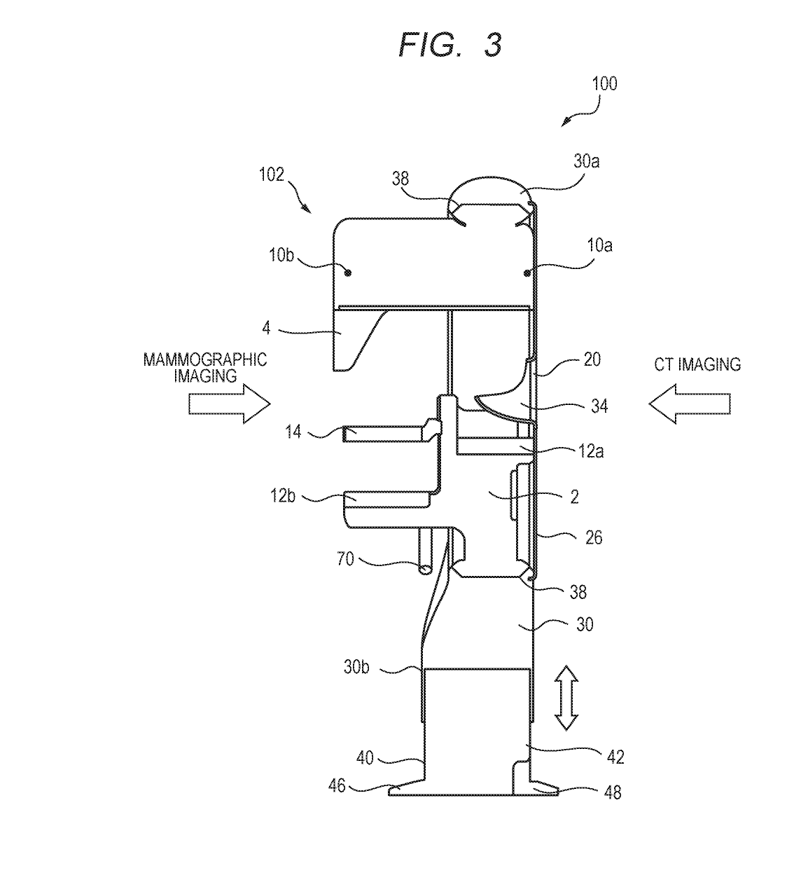

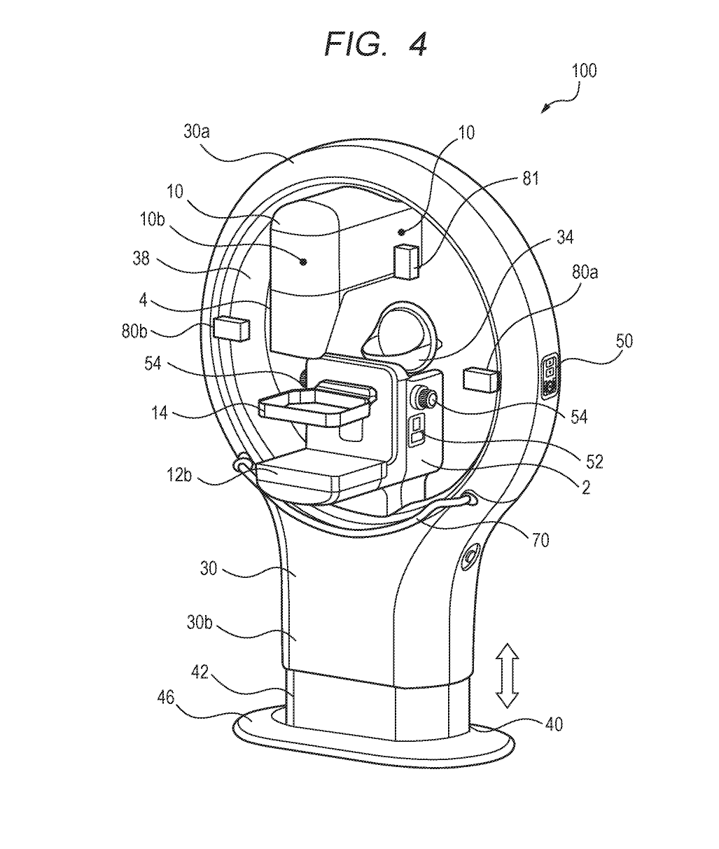

The breast imaging system 100 is now described in more detail with reference to FIG. 2 to FIG. 4. FIG. 2 is a view for illustrating the appearance of the breast imaging system 100 as viewed from the CT imaging side. FIG. 3 is a cross-sectional view of the breast imaging system 100. The cross-sectional view of the breast imaging system 100 is a cross-sectional view taken along the center line (dash-dot line) extending in a vertical direction of the breast imaging system 100 of FIG. 2. FIG. 4 is a view for illustrating the appearance of the breast imaging system 100 as viewed from the mammographic imaging side.

As illustrated in FIG. 2, the front cover 26 for protecting the subject from the radiation generation unit 10 and the radiation detection unit 12 that rotate during CT imaging is arranged on the gantry 30 on the CT imaging side. The front cover 26 has the opening 20 through which the breast of the subject on which CT imaging is to be performed is inserted. The front cover 26 is seamlessly arranged so that there is no boundary line between the opening 20 and the breast cup (breast holder) 34.

As illustrated in FIG. 4, the compression plate for compressing the breast of the subject on which mammographic imaging is to be performed is arranged on the gantry 30 on the mammographic imaging side. A protective plate 4 for protecting the subject from unnecessary radiation is also arranged on the gantry 30 on the mammographic imaging side.

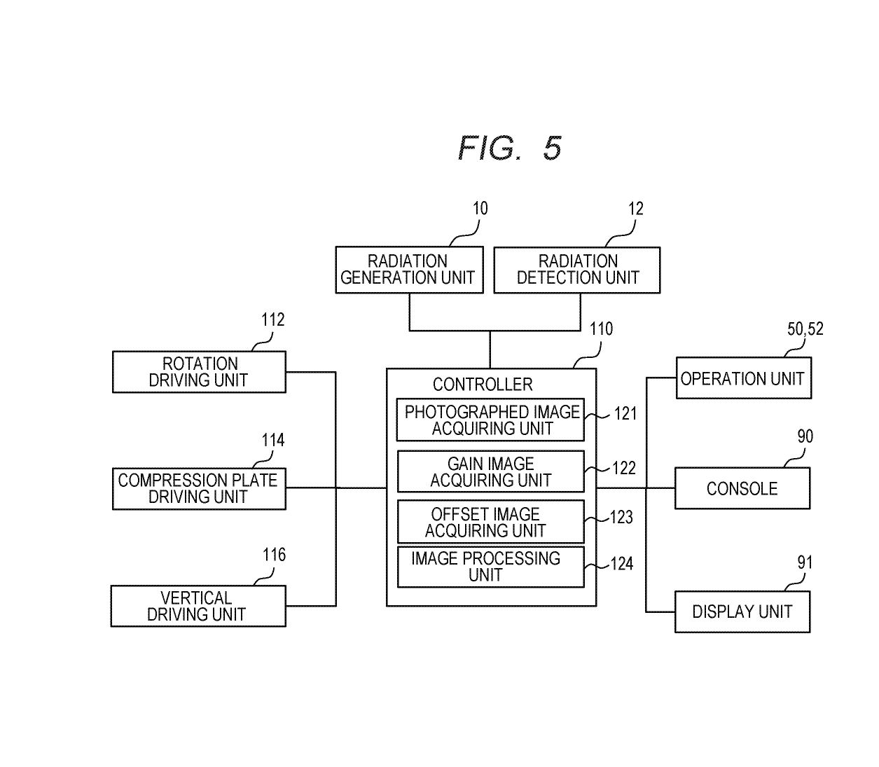

In FIG. 5, there is illustrated an example of a configuration diagram of the radiation breast imaging system 100. The breast imaging system 100 includes the radiation generation unit 10, the radiation detection unit 12, and a rotation driving unit 112 configured to rotate the radiation generation unit 10 and the radiation detection unit 12 under a state in which the radiation generation unit 10 and the radiation detection unit 12 face each other. The breast imaging system 100 also includes a compression plate driving unit 114 configured to vertically move the compression plate 14 and a vertical driving unit 116 configured to vertically move the gantry 30 with respect to the support leg 40.

The breast imaging system 100 includes a controller 110 configured to control each of the radiation generation unit 10, the radiation detection unit 12, the rotation driving unit 112, the compression plate driving unit 114, and the vertical driving unit 116. The breast imaging system 100 also includes operation units 50 and 52 and a console 90, which are configured to transmit instructions to the controller 110, and a display unit 91 configured to display radiation images (including a photographed image, an image for gain calibration, and an offset image).

The operation unit 50 configured to operate the breast imaging system 100 is arranged on the gantry 30. The operation unit 52 having the same function as the operation unit 50 is arranged on a support stage 2 configured to support the radiation detection unit 12. The console 90 is arranged externally to an imaging room.

The radiation generation unit 10 mainly includes a target (not shown) and an electron emission source (not shown) configured to generate electrons. Electrons generated by the electron emission source are emitted toward the target side due to a potential difference between a cathode and an anode of the radiation generation unit 10. The target is a member configured to generate radiation as a result of electrons colliding with the target. The radiation emitted from the target is formed into a cone beam shape, and is radiated toward the outside. The controller 110 is capable of controlling imaging conditions of the radiation generation unit 10.

The radiation detection unit 12 is configured to detect by a photoelectric conversion element the radiation that has passed through the subject, and to output the detected radiation as an electric signal. For example, the radiation detection unit 12 includes a conversion panel configured to detect the radiation that has passed through the subject, an electricity storage unit, and an interface (I/F) for outputting information obtained by converting the radiation into an electric signal. The electric signal is output to the controller 110 by the I/F.

The controller 110 includes a photographed image acquiring unit 121, a gain image acquiring unit 122, an offset image acquiring unit 123, and an image processing unit 124. The photographed image acquiring unit 121 is configured to acquire a radiation image (i.e., photographed image: second radiation image) photographed by irradiating the radiation detection unit 12 with radiation from the radiation generation unit 10 under a state in which the subject is in an imaging range.

The gain image acquiring unit (i.e., image acquiring unit) 122 is configured to acquire an image for gain calibration (i.e., first radiation image) photographed by irradiating the radiation detection unit 12 with radiation from the radiation generation unit 10 under a state in which the subject is not in the imaging range (i.e., without the subject). The offset image acquiring unit 123 is configured to acquire an offset image photographed without irradiating the radiation detection unit 12 with radiation from the radiation generation unit 10.

The image processing unit 124 is configured to perform, on the photographed image, sensitivity correction (gain calibration, offset calibration, etc.) for each pixel based on information on the image for gain calibration and the offset image. The image processing unit 124 is also configured to generate a mammographic image for diagnosis. The image processing unit 124 is also configured to perform a CT reconstruction process to generate a CT image for diagnosis by using an image for reconstruction. Each component 121 to 124 in the controller 110 can be realized by software modules which are excused by a processor, or any circuits (e.g., application specific integrated circuit (ASIC)).

<Gantry>

As illustrated in FIG. 2 to FIG. 4, the gantry 30 includes the ring-shaped rotating frame 38 for rotating the radiation generation unit 10 and the radiation detection unit 12 under a state in which the radiation generation unit 10 and the radiation detection unit 12 face each other, and a ring-shaped fixed frame 30a for rotatably supporting the rotating frame 38. The gantry 30 also includes an oblong cylindrical portion 30b that has an oblong cylindrical shape and that is connected to the fixed frame 30a. The rotating frame 38 and the fixed frame 30a may be referred to as a rotation unit configured to rotate the radiation generation unit 10 and the radiation detection unit 12.

The fixed frame 30a and the oblong cylindrical portion 30b are integrally formed. The fixed frame 30a is positioned above the oblong cylindrical portion 30b. The oblong cylindrical portion 30b is connected to the support leg 40 supporting the gantry 30 on the floor surface.

The gantry 30 stands upright in the vertical direction so that imaging can be performed on the subject in a standing position. The axis of rotation of the rotation unit (i.e., the axis of rotation of the rotating frame 38 in the gantry 30) configured to rotate the radiation generation unit 10 and the radiation detection unit 12 is the horizontal direction.

The oblong cylindrical portion 30b covers an outer circumference of an oblong cylindrical portion 42 of the support leg 40. More specifically, the oblong cylindrical portion 42 of the support leg 40 is arranged inside the oblong cylindrical portion 30b of the gantry 30, and the oblong cylindrical portion 42 of the support leg 40 and the oblong cylindrical portion 30b of the gantry 30 form a nested structure.

The breast imaging system 100 includes the vertical driving unit 116 configured to vertically move the oblong cylindrical portion 30b with respect to the support leg 40, and is capable of adjusting the height of the opening 20 in accordance with the body shape of the subject.

<Radiation Generation Unit and Radiation Detection Unit>

The breast imaging system 100 includes the radiation generation unit 10, which includes a radiation tube configured to generate radiation, and the radiation detection unit 12, which includes a radiation detector configured to detect the radiation radiated from the radiation generation unit 10. The breast imaging system 100 is capable of rotating the radiation generation unit 10 and the radiation detection unit 12 under a state in which the radiation generation unit 10 and the radiation detection unit 12 face other.

The radiation generation unit 10 and the radiation detection unit 12 are arranged on the rotating frame 38, which is configured to rotate with respect to the fixed frame 30a of the gantry 30. As illustrated in FIG. 3, the breast imaging system 100 includes a radiation generation unit 10a and a radiation detection unit 12a for CT imaging, and a radiation generation unit 10b and a radiation detection unit 12b for mammographic imaging. The gantry 30 includes the radiation generation unit 10a and the radiation detection unit 12a for CT imaging, and the radiation generation unit 10b and the radiation detection unit 12b for mammographic imaging. In other words, the breast imaging system 100 includes two pairs of radiation generation units and radiation detection units for CT imaging and mammographic imaging.

The gantry 30 includes the ring-shaped rotating frame 38 for rotating the radiation generation unit 10a and the radiation detection unit 12a for CT imaging under a state in which those units face each other, and for rotating the radiation generation unit 10b and the radiation detection unit 12b for mammographic imaging under a state in which those units face each other.

Specifically, for CT imaging, the radiation generation unit 10a and the radiation detection unit 12a are arranged on the rotating frame 38. The radiation detection unit 12a is arranged on the rotating frame 38 via the support stage 2 configured to support the radiation detection unit 12a.

For mammographic imaging, the radiation generation unit 10b and the radiation detection unit 12b are arranged on the rotating frame 38. The radiation detection unit 12b is arranged on the rotating frame 38 via the support stage 2.

The rotating frame 38 is connected to the fixed frame 30a of the gantry 30 via a bearing having a bearing structure. The fixed frame 30a is a stationary frame that is in an immobile state. The rotating frame 38 can be rotated by the rotation driving unit 112. The rotation driving unit 112 is arranged in the gantry 30 such that the axis of rotation of the rotating frame 38 is in the horizontal direction.

The compression plate 14 is arranged in a vertically movable manner on the support stage 2. A rotation knob 54 for vertically moving the compression plate 14 is arranged on the support stage 2. The breast of the subject can be sandwiched between the compression plate and the radiation detection unit 12b by rotating the rotation knob 54 to lower the compression plate 14.

In this way, the support stage 2 is arranged on the rotating frame 38, and is configured to support the radiation detection unit 12a, the radiation detection unit 12b, and the compression plate 14. Rotating the rotating frame 38 together with the support stage 2 by the rotation driving unit 112 enables the radiation detection unit 12a and the radiation detection unit 12b to be rotated. Rotating the rotating frame 38 by the rotation driving unit 112 enables the radiation generation unit 10a and the radiation generation unit 10b to be rotated.

As illustrated in FIG. 3, the radiation generation unit 10a and the radiation generation unit 10b are arranged at substantially the same height. The radiation detection unit 12a is arranged at a higher position than the radiation detection unit 12b.

In other words, the radiation generation unit 10a and the radiation generation unit 10b are arranged such that the radiation generation unit 10a and the radiation generation unit 10b are at the same position with respect to (i.e., the same distance from) the axis of rotation of the rotation unit (i.e., rotating frame 38).

The radiation detection unit 12a and the radiation detection unit 12b are arranged such that the radiation detection unit 12b is positioned closer to the external side than the radiation detection unit 12a with respect to the axis of rotation of the rotation unit (i.e., rotating frame 38).

The distance between the radiation generation unit 10a and the radiation detection unit 12a to be used when CT imaging is performed is shorter than the distance between the radiation generation unit 10b and the radiation detection unit 12b to be used when mammographic imaging is performed.

When mammographic imaging is performed, the breast of the subject is sandwiched between the compression plate 14 and the radiation detection unit 12b. The breast of the subject is squashed flat from the compression by the compression plate 14 and the radiation detection unit 12b, and hence it is necessary to secure a field of view (FOV) by increasing the surface area on which radiation is irradiated. In order to achieve this, the radiation detection unit 12b to be used when mammographic imaging is performed is arranged at a lower position than the radiation detection unit 12a to be used when CT imaging is performed.

The FOV by the radiation generation unit 10b for mammographic imaging is referred to as a first FOV. The radiation generation unit 10b and the radiation detection unit 12b are arranged such that the first FOV from the radiation generation unit 10b includes the compression plate 14. The first FOV has a square pyramid shape (cone beam shape) that spreads from a focal point of the radiation generation unit 10b as an apex.

The radiation generation unit 10a and the radiation detection unit 12a are arranged such that, when CT imaging is performed, the size of the rotating frame 38 and the overall size of the breast imaging system 100 (i.e., gantry 30) are compact. Specifically, the distance between the radiation generation unit 10a and the radiation detection unit 12a is set to be as short as possible. The radiation detection unit 12a is arranged directly below the breast holder 34. The radiation detection unit 12a is arranged at a position at which the radiation detection unit 12 does not contact the breast holder 34 even when the radiation detection unit 12a is rotated by the rotating frame 38.

The FOV by the radiation generation unit 10a for CT imaging is referred to as a second FOV. The breast of the subject on which CT imaging is to be performed is held on the breast holder 34, and is not compressed. The radiation generation unit 10a and the radiation detection unit 12a are arranged such that the second FOV from the radiation generation unit 10a includes a tip portion of the breast holder 34. The second FOV has a square pyramid shape (cone beam shape) that spreads from a focal point of the radiation generation unit 10a as an apex.

It is possible for breast cancer to spread to the breast surroundings (axilla). The first FOV of the radiation generation unit 10b for mammographic imaging and the second FOV of the radiation generation unit 10a for CT imaging are each set such that an image of the surroundings of the breast (axilla) of the subject can be photographed.

When CT imaging is performed, the radiation generation unit 10a for CT imaging may be arranged at a higher position than the radiation generation unit 10b for mammographic imaging in order to secure the FOV. When CT imaging is performed, the radiation generation unit 10a and the radiation detection unit 12a are rotated while the radiation generation unit 10a is generating radiation from the focus point.

In this way, the breast imaging system 100 of this embodiment includes a first radiation generation unit 10b configured to generate radiation, a second radiation generation unit 10a configured to generate radiation, a first radiation detection unit 12b configured to detect radiation radiated from the first radiation generation unit 10b, and a second radiation detection unit 12a configured to detect radiation radiated from the second radiation generation unit 10a.

Imaging is performed from the first side of the breast imaging system 100 by using the first radiation generation unit 10b and the first radiation detection unit 12b under a state in which the site to be imaged of the subject is sandwiched between the compression plate 14 and the first radiation detection unit 12b. Imaging is performed from the second side, which is opposite from the first side, of the breast imaging system 100 by rotating the second radiation generation unit 10a and the second radiation detection unit 12a under a state in which the site to be imaged of the subject is inserted between the second radiation generation unit 10a and the second radiation detection unit 12a.

As described above, the breast imaging system 100 includes two pairs of radiation generation units and radiation detection units for CT imaging and for mammographic imaging. Therefore, an FOV can be secured that is suited to each of the breast of the subject on which CT imaging is to be performed and that is suited to the breast of the subject on which mammographic imaging is to be performed.

<Rotating Frame>

The breast imaging system 100 includes the rotation driving unit 112 configured to rotate the radiation generation unit 10 and the radiation detection unit 12 via the rotating frame 38. The radiation generation unit 10 includes the radiation generation unit 10b for mammographic imaging and the radiation generation unit 10a for CT imaging.

In FIG. 4, there is illustrated a mode in which craniocaudal (CC) view mammographic imaging is performed by the breast imaging system 100. The position of the rotating frame 38 is set such that the radiation generation unit 10b, the compression plate 14, and the radiation detection unit 12b are aligned in the vertical direction.

The distance between the compression plate 14 and the radiation detection unit 12b can be adjusted by rotating the rotation knob 54 to move the compression plate 14. The breast of the subject can be compressed by moving the compression plate 14. In the CC mammographic imaging illustrated in FIG. 4, the breast positioned between the compression plate 14 and the radiation detection unit 12b is compressed between the compression plate 14 and the radiation detection unit 12b, and a radiation image of the breast is photographed.

The rotation driving unit 112 is arranged in an interior portion of the fixed frame 30a. The rotating frame 38 is rotatably connected to the rotation driving unit 112 via a connection member (e.g., belt). A bearing is set in a gap between the fixed frame 30a and the rotating frame 38. The rotating frame 38 is rotated about the fixed frame 30a based on driving by the rotation driving unit 112.

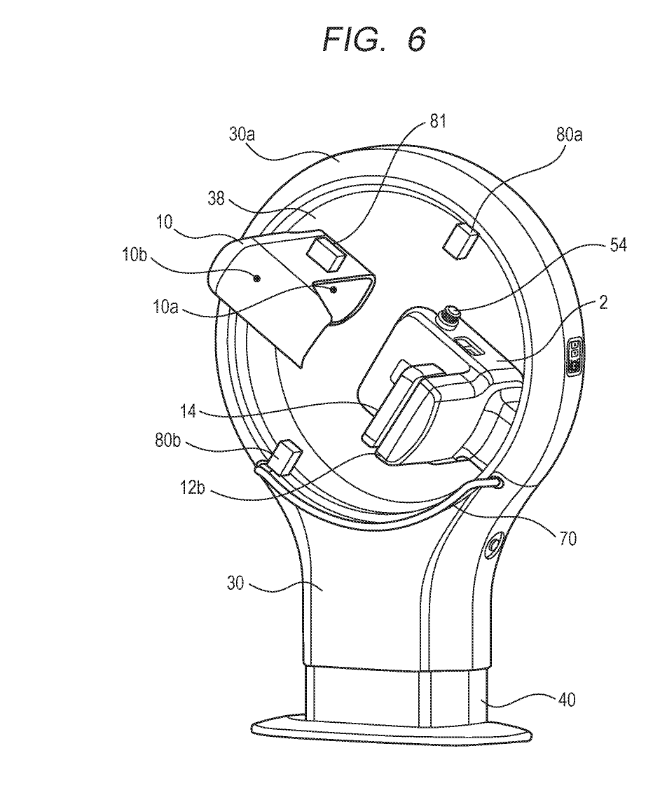

FIG. 6 is a view for illustrating a rotation mode in which the radiation generation unit 10b and the radiation detection unit 12b have been rotated by the rotation driving unit 112 of the breast imaging system 100. When mediolateral oblique (MLO) view mammographic imaging is to be performed by the breast imaging system 100, as illustrated in FIG. 6, the rotating frame 38 is rotated from the state of FIG. 4 by a predetermined angle (e.g., 65 degrees), and is then stopped. The stationary state of the rotating frame 38 may be maintained by a servo or by a brake.

In the MLO mammographic imaging illustrated in FIG. 6, the breast positioned between the compression plate and the radiation detection unit 12b is compressed between the compression plate 14 and the radiation detection unit 12b, and then is imaged with the radiation.

When CT imaging is to be performed, the rotating frame 38 is rotated about the fixed frame 30a based on driving by the rotation driving unit 112. Specifically, the rotating frame 38 is rotated by at least 180 degrees. During the rotation of the rotating frame 38, the radiation generation unit 10a generates radiation, and the radiation detection unit 12a detects the radiation.

The radiation detection unit 12 is capable of performing CT imaging on the breast of the subject by detecting the radiation that has passed through the breast of the subject. The breast imaging system 100 is capable of generating a CT image by reconstructing the radiation data acquired by the imaging.

<Front Cover>

The front cover 26 is detachably mountable to the gantry 30 of the breast imaging system 100. In other words, the front cover 26 is detachably mountable to the ring-shaped fixed frame 30a. Specifically, the front cover 26 is mounted to the fixed frame 30a by fitting a protruding portion of the front cover 26 to a frame body (i.e., to a groove) of the fixed frame 30a.

In FIG. 2, there is illustrated a mode in which the front cover 26 is mounted to the gantry 30 of the breast imaging system 100.

The front cover 26 may be removed from the gantry 30 of the breast imaging system 100. When the front cover 26 is removed from the gantry 30, the breast of the subject on which mammographic imaging is to be performed can be accessed by an operator from the CT imaging side via a hollow portion of the rotating frame 38. During mammographic imaging, the position of the breast positioned between the radiation detection unit 12b and the compression plate 14 of the breast imaging system 100 and the pressure being applied on the breast may be adjusted.

The fixed frame 30a of the gantry 30 has a ring shape, and hence the front cover 26 is circular. The front cover 26 may be fixed to a member that is immobile with respect to the rotation of the radiation generation unit 10 and the radiation detection unit 12.

The opening 20 for inserting the breast of the subject is arranged on the front cover 26. Specifically, as illustrated in FIG. 2, the circular opening 20 for inserting the breast of the subject is arranged in a center portion of the front cover 26.

The front cover 26 is constructed from a shielding member configured to shield radiation. During CT imaging or mammographic imaging, scattered radiation does not pass through the front cover 26. In other words, scattered radiation does not reach the CT imaging side. The front cover 26 is a semitransparent member. The front cover 26 is semitransparent, and hence the position of the radiation generation unit 10 and the radiation detection unit 12 can be confirmed from the CT imaging side even when the front cover 26 is arranged on the gantry 30.

As illustrated in FIG. 3 and FIG. 4, the breast holder 34 is arranged on the front cover 26 as a holding stage for holding the breast inserted from the opening 20. The breast holder 34 is a transparent member through which radiation passes. The breast holder 34 is formed along a periphery of the opening 20 of the front cover 26. The breast holder 34 is formed protruding toward the imaging unit 102 side from the opening 20 of the front cover 26.

The breast holder 34 is hollow, and is curved so as to match the shape of the breast. A part (lower side) of the breast holder 34 has a bowl shape, and a part (upper side) of the breast holder 34 is open. The breast holder 34 has an opening that allows the operator to access the breast from the mammographic imaging side.

The breast holder 34 has a shape for holding the lower side of the breast. The breast holder 34 is capable of holding the breast while maintaining the shape of the breast. The breast holder 34 does not compress the breast.

As illustrated in FIG. 4, a part (upper side) of the breast holder 34 is open, and hence the operator can access the breast of the subject on which CT imaging is to be performed from the mammographic imaging side. Specifically, the operator can access the breast of the subject that is held in the breast holder 34 from the mammographic imaging side.

The breast holder 34 is detachably mountable to the front cover 26. Various sizes of breast holder 34 may be prepared. Specifically, the operator can prepare a plurality of breast holders 34. For example, the breast holder 34 may be replaced in accordance with the size of the breast of the subject.

The front cover 26 and the breast holder 34 fixed to the gantry 30 separate the space of the subject from the radiation generation unit 10 and the radiation detection unit 12 that rotate during CT imaging. The breast of the subject is held in the breast holder 34, and hence is fixed during imaging.

An example has been described in which the breast holder 34 is connected along the periphery of the opening 20 of the front cover 26. However, the present invention is not limited to this example. For example, the breast holder 34 may be connected to the fixed frame 30a of the gantry 30.

The front cover 26 may be non-transparent from the side of the subject on which CT imaging is to be performed, and be transparent from the operator side of the mammographic imaging side. When the front cover 26 is non-transparent from the subject side, fear arising from the movement of the radiation generation unit 10 and the radiation detection unit 12 seen through the front cover 26 can be avoided.

<Calibration>

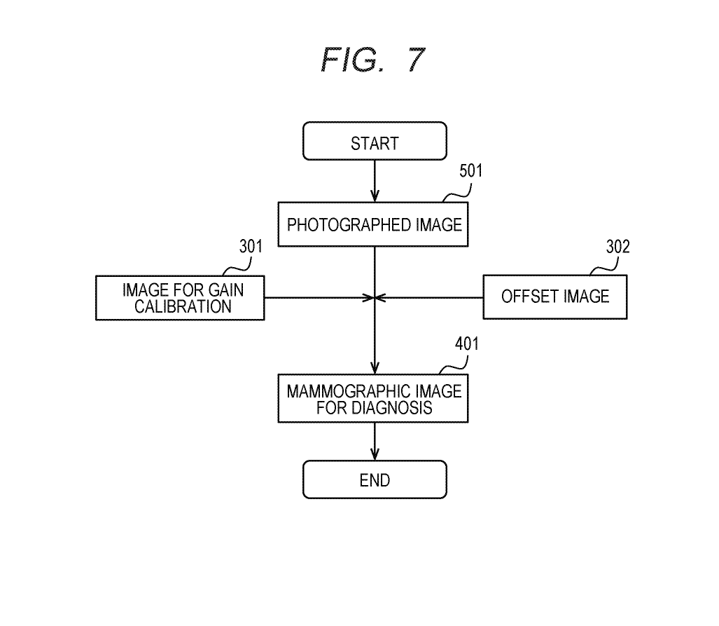

In FIG. 7, there is illustrated an example of a process configuration for mammographic image correction of this embodiment. When mammographic imaging is to be performed, in order to perform sensitivity correction of the radiation detection unit 12b for mammographic imaging before imaging is performed on the subject, an image for gain calibration 301 is collected by irradiating the radiation detection unit 12b with radiation from the radiation generation unit 10b under a state in which there is no subject and collecting a signal from each pixel. An offset image 302 is collected by collecting a signal from each pixel of the radiation detection unit 12b under a state in which radiation is not radiated from the radiation generation unit 10b.

When for some reason at least one of the image for gain calibration 301 and the offset image 302 failed to be collected before imaging is performed on the subject, the missing data can be collected after imaging is performed on the subject.

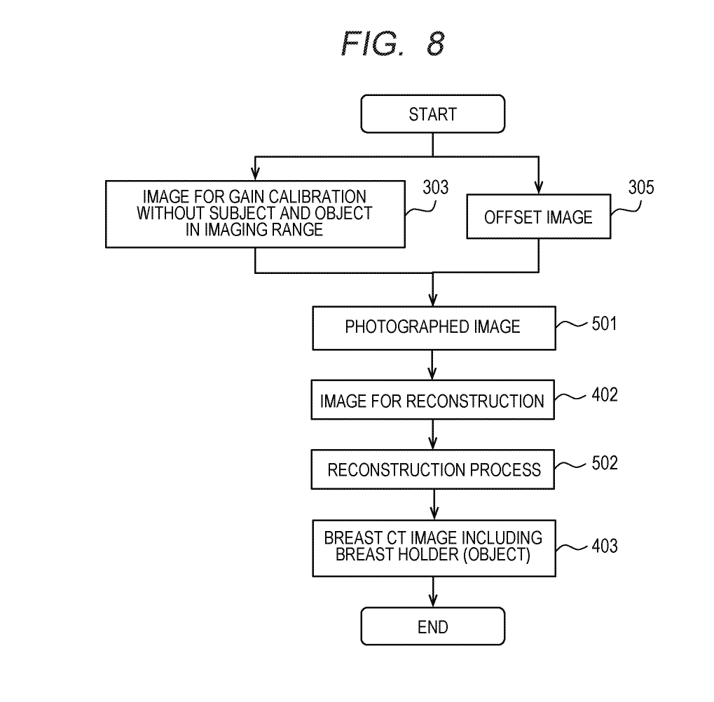

In FIG. 8, there is illustrated an example of a process configuration for CT image correction of this embodiment. When CT imaging is to be performed, sensitivity correction of the radiation detection unit 12a for CT imaging is performed before imaging is performed on the subject. For that reason, the sensitivity correction is performed under a state where a subject is not in the imaging range and the breast holder 34 is detached from the front cover 26. When the breast holder 34 cannot be detached from the front cover, the breast holder 34 may be detached from the apparatus together with the front cover.

The radiation generation unit 10a and the radiation detection unit 12a are then rotated 360 degrees in line with the CT imaging while radiation is radiated continuously or intermittently in pulses on the radiation detection unit 12a from the radiation generation unit 10a. The gain image acquiring unit 122 collects an image for gain calibration 303 at all rotation angles.

During this operation, the collection angle does not need to be the same angle range or the same interval as those of the actual CT imaging. The data used for the process may also be a single piece of data collected at an arbitrary angle within the 360 degrees.

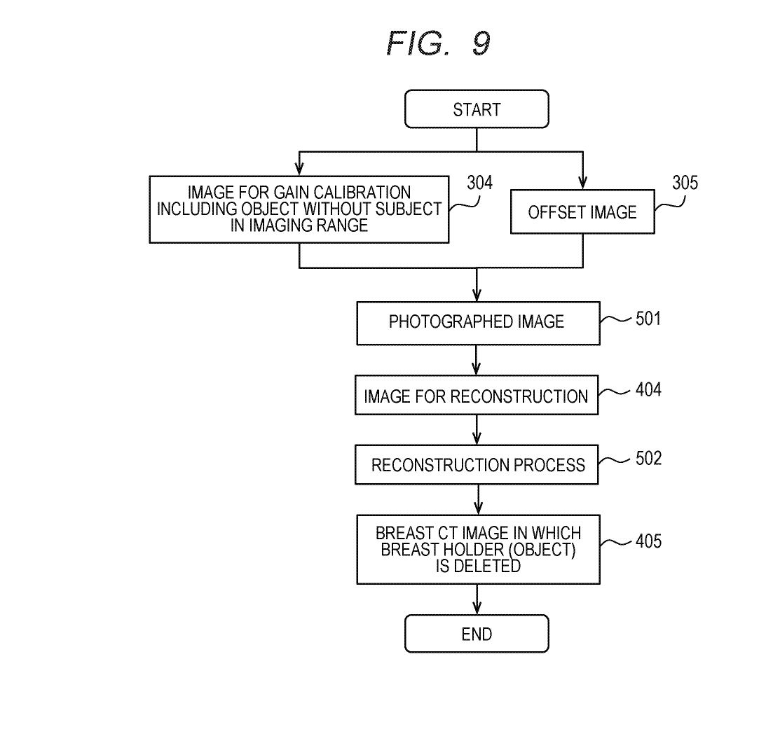

In FIG. 9, there is illustrated an example of a process configuration for CT image correction for deleting the breast holder 34 of this embodiment. The breast holder 34 is fixed to the front cover 26 under a state in which the subject (i.e., breast) is not held and the breast holder 34 is included in the imaging range. Then, the radiation generation unit 10a and the radiation detection unit 12a are rotated 360 degrees in line with the CT imaging while radiation is radiated continuously or intermittently in pulses on the radiation detection unit 12a from the radiation generation unit 10a. The gain image acquiring unit 122 collects an image for gain calibration 304 including the breast holder 34 for all rotation angles.

The gain image acquiring unit (i.e., image acquiring unit) 122 acquires the image for gain calibration (i.e., first radiation image) 304 to be photographed by irradiating the breast holder (i.e., object) with radiation under a state in which the subject is not in the imaging range (i.e., without the subject). The image for gain calibration (i.e., first radiation image) 304 is acquired by radiating the radiation from the radiation generation unit 10, which is rotating about the breast holder (i.e., object) 34, at an arbitrary angle. The breast holder (i.e., object) is to be deleted from the image.

During this operation, the collection angle does not need to be the same angle range or the same interval as those of the actual CT imaging. The data used for the process may also be a single piece of data collected at an arbitrary angle within the 360 degrees.

When there are a plurality of breast holders 34, the image for gain calibration 304 including a breast holder is similarly collected for all of the breast holders 34 that are planned to be used. In this case, the gain image acquiring unit (i.e., image acquiring unit) 122 acquires a plurality of first radiation images of the plurality of breast holders (i.e., objects) 34. The image processing unit 124 selects the first radiation image corresponding to the object in the photographed image from among the plurality of first radiation images, and corrects the photographed image by using the selected first radiation image.

As illustrated in FIG. 8 and FIG. 9, under a state in which radiation is not being radiated from the radiation generation unit 10a, the offset image acquiring unit 123 acquires an offset image 305 by collecting a signal from each pixel of the radiation detection unit 12a. The offset image 305 is collected at all rotation angles by rotating the radiation generation unit 10a and the radiation detection unit 12a 360 degrees in line with the CT imaging. During this operation, it is not necessary for the collection angle to be the same angle range or the same interval as those of the actual CT imaging. The data used for the process may also be a single piece of data collected at an arbitrary angle within the 360 degrees.

When for some reason at least one of the image for gain calibration 303, the image for gain calibration 304 including a breast holder, and the offset image 305 failed to be collected before imaging is performed on the subject, the missing data can be collected after imaging is performed on the subject.

<Correction Image Generation>

Sensitivity correction is performed on an image (i.e., second radiation image) 501 photographed from the subject based on data collected by calibration.

As illustrated in FIG. 7, in mammographic imaging, the image processing unit 124 performs sensitivity correction on the image (i.e., second radiation image) 501 actually photographed from the subject for each pixel based on information on the image for gain calibration 301 and the offset image 302, to thereby generate a mammographic image 401 for diagnosis.

In the CT imaging of FIG. 8, the photographed image acquiring unit 121 acquires the photographed image 501 by rotating the radiation generation unit 10a and the radiation detection unit 12a 360 degrees, and continuously radiating radiation or intermittently radiating radiation in pulses on the subject. The photographed image acquiring unit (i.e., image acquiring unit) 121 acquires the image (i.e., second radiation image) 501 that is photographed by radiating radiation under a state in which the subject and the breast holder (i.e., object) 34 are in the imaging range.

The image processing unit 124 performs sensitivity correction on the photographed image 501 for each pixel based on information on the image for gain calibration 303 and the offset image 305. Then, the image processing unit 124 generates an image 402 for reconstruction, which is a continuous 360-degree rotating image.

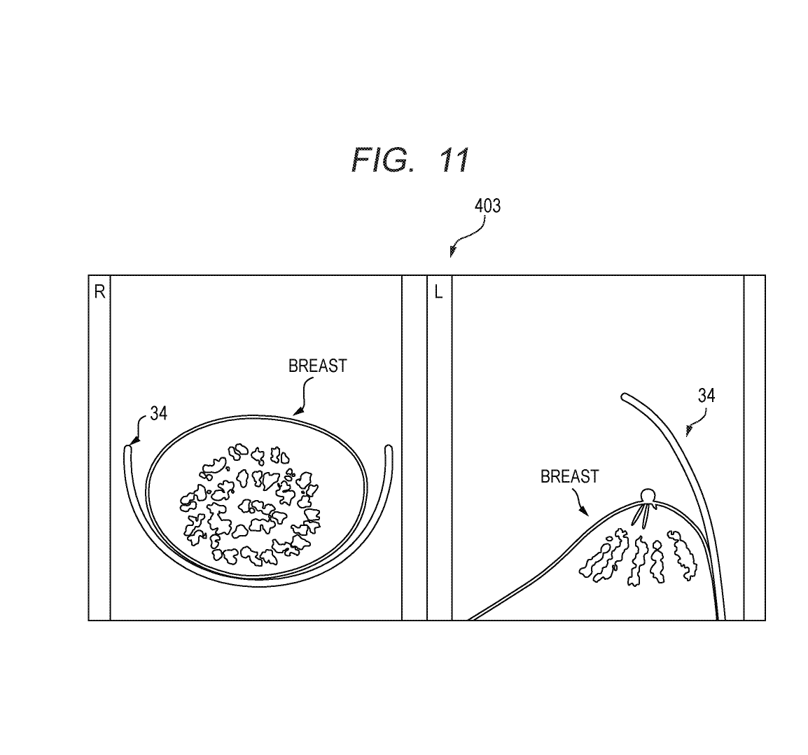

After that, the image processing unit 124 performs a CT reconstruction process 502 by using the image 402 for reconstruction, and a CT image 403 for diagnosis illustrated in FIG. 11 is generated. For the CT image 403 for diagnosis, the pixel values are calculated so that the image includes the breast holder 34 and the imaging site (i.e., breast).

In the CT imaging of FIG. 9, the photographed image (i.e., second radiation image) 501 is acquired by rotating the radiation generation unit 10a and the radiation detection unit 12a 360 degrees, and continuously radiating radiation or intermittently radiating radiation in pulses on the subject. The photographed image 501 is acquired by radiating radiation from the radiation generation unit 10, which is rotating about the subject and the breast holder (i.e., object) 34, at an arbitrary angle.

The image processing unit 124 performs sensitivity correction on the photographed image 501 for each pixel based on information on the image for gain calibration (i.e., first radiation image) 304, which includes the breast holder 34 under a state in which there is no subject (i.e., breast), and the offset image 305. The image processing unit 124 performs correction for deleting the image information on the breast holder (i.e., object) 34 from the photographed image (i.e., second radiation image) 501 by using the image for gain calibration (i.e., first radiation image) 304. Then, the image processing unit 124 generates an image 404 for CT reconstruction, which is a continuous 360-degree rotating image.

In this way, the image processing unit 124 corrects the photographed image (i.e., second radiation image) 501 acquired by radiating radiation under a state in which the subject and the breast holder (i.e., object) 34 are in the imaging range by using the image for gain calibration (i.e., first radiation image) 304. The image processing unit 124 also performs offset calibration on the photographed image 501 by using the offset image 305.

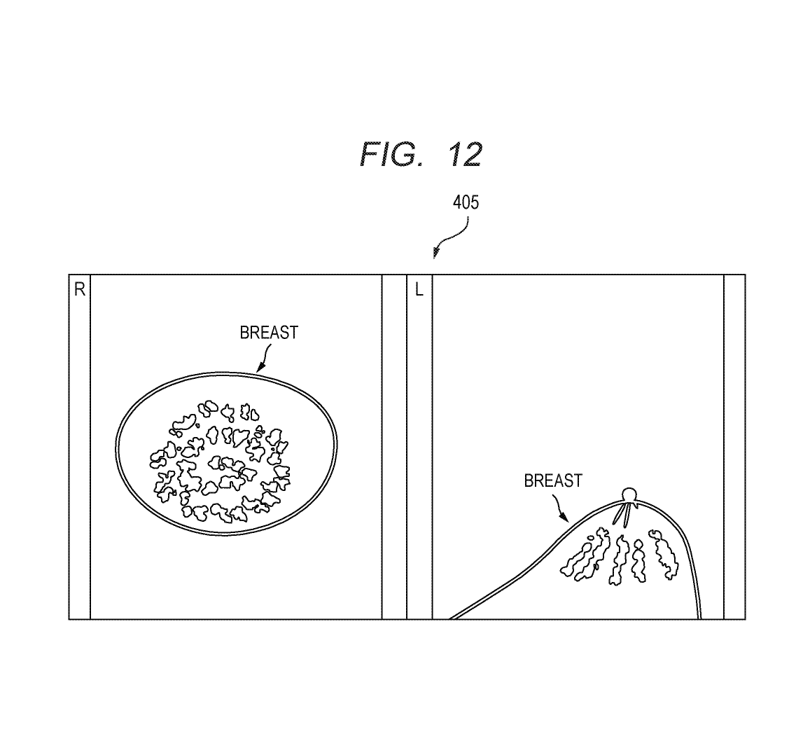

After that, the image processing unit 124 performs the CT reconstruction process 502 by using the image 404 for reconstruction, to thereby generate a CT image 405 for diagnosis illustrated in FIG. 12. For the CT image 405 for diagnosis, the pixel values are calculated so that the image includes the imaging site (i.e., breast) under a state in which the breast holder 34 has been deleted. In this way, the image processing unit 124 corrects the photographed image (second radiation image) 501 by using the first radiation image, to thereby delete the breast holder (i.e., object) 34 from the photographed image 501.

For the CT image 405 for diagnosis, the pixel values may be calculated so that the image includes the imaging site (i.e., breast) under a state in which the pixel values of the breast holder 34 have been adjusted (e.g., state in which the breast holder 34 is semitransparent). In this case, the image processing unit 124 adjusts the pixel values of the first radiation image to correct the photographed image 501.

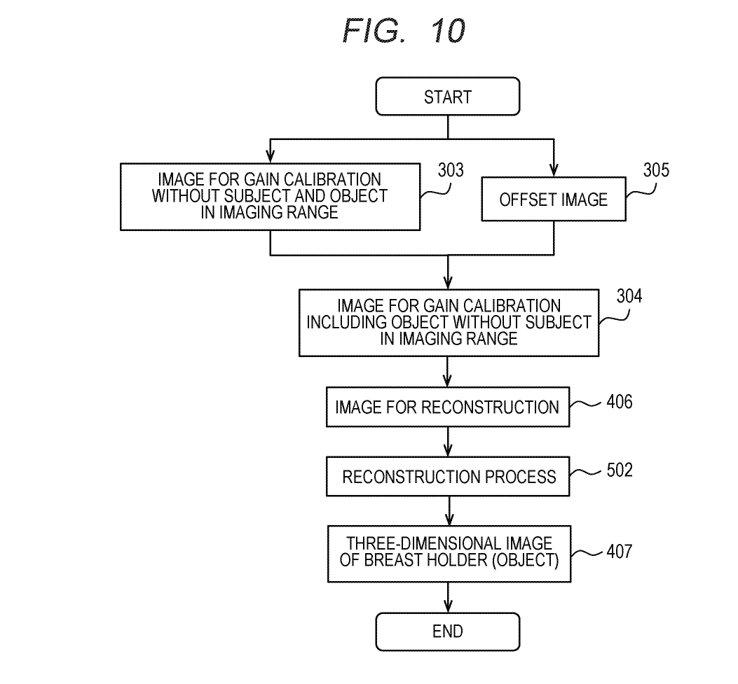

In FIG. 10, there is illustrated an example of a process configuration for CT image correction of the breast holder 34 of this embodiment. The image processing unit 124 performs sensitivity correction on, as the photographed image, the image for gain calibration 304 including the breast holder 34 under a state in which there is no subject (i.e., breast) based on information on the image for gain calibration 303 and the offset image 305. Then, an image 406 for CT reconstruction of the breast holder 34 is generated.

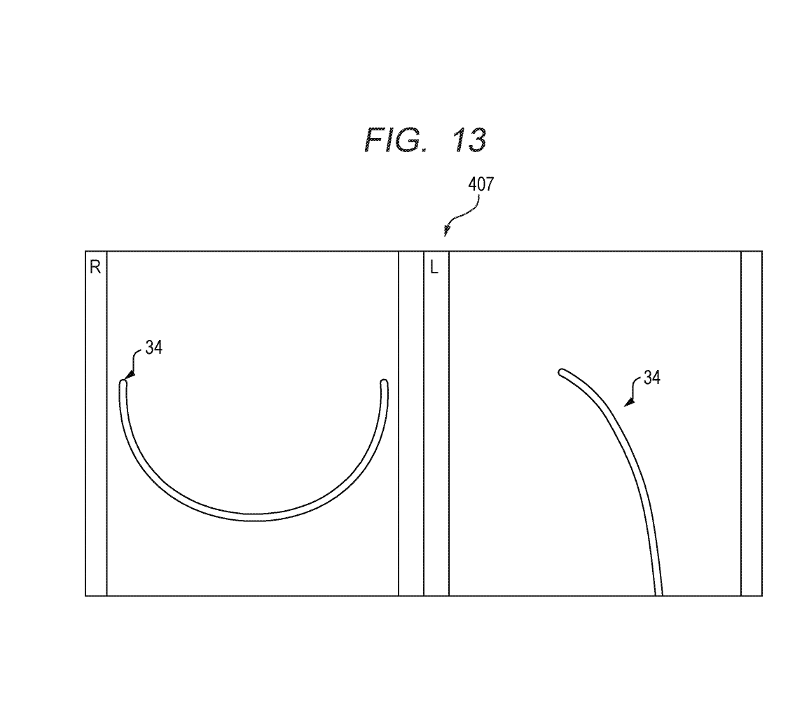

The image processing unit 124 performs the CT reconstruction process 502 by using the image 406 for CT reconstruction of the breast holder 34. Thus, as illustrated in FIG. 13, an image 407 of the breast holder 34 under a state in which the subject (i.e., breast) is not held is reconstructed.

<CT Image Display>

When the CT-reconstructed image is displayed, diagnosis is performed by displaying the CT image 405 for diagnosis including the imaging site (i.e., breast) under a state in which the breast holder 34 has been deleted. When a positional relation between the breast holder 34 and the imaging site is required, the CT image 405 for diagnosis and the CT image 403 for diagnosis including the breast holder 34 and the imaging site (i.e., breast) may be switched and displayed. The display unit 91 switches and displays the second radiation image (i.e., CT image 403 for diagnosis) and the second radiation image (i.e., CT image 405 for diagnosis) corrected by using the first radiation image.

In this case, in place of the CT image 403 for diagnosis, a combined image may be displayed in which the image 407 of the breast holder 34 under a state in which the subject (i.e., breast) is not held overlaps the CT image 405 for diagnosis including the imaging site (i.e., breast) under a state in which the breast holder 34 has been deleted. The display unit 91 generates the combined image by combining the object (i.e., image 407) of the first radiation image with the second radiation image (i.e., CT image 405 for diagnosis) corrected by using the first radiation image, and switches and displays the second radiation image corrected by using the first radiation image and the combined image.

The image 407 of the breast holder 34 under a state in which the subject (i.e., breast) is not held may be used in a shared manner when an image of another subject (i.e., breast) when the same breast holder 34 is used is displayed.

The embodiment of the present invention has been described above, but the present invention is not limited thereto, and changes and modifications can be made thereto within the scope of the appended claims.

In this embodiment, the image for gain calibration 304 is collected such that the object is included in the imaging range under a state in which there is no subject, and the CT image 405 for diagnosis in which the object has been deleted from the photographed image 501 of the subject and the object is generated by using the image for gain calibration 304. In this embodiment, the breast holder 34 is deleted, but the CT image 405 for diagnosis may also be generated by deleting a biopsy needle or other such object.

In this embodiment, a radiation imaging apparatus capable of executing mammographic imaging and CT imaging has been described, but the present invention is also applicable to a radiation imaging apparatus for CT imaging.

According to the present invention, an image in which an object has been deleted can be obtained by correcting a photographed image by using a radiation image acquired in advance by irradiating the object with radiation under a state in which a subject is not in the imaging range.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-173301, filed Sep. 6, 2016 which is hereby incorporated by reference herein in its entirety.

What is claimed is:

1. A radiation imaging apparatus, comprising:
an image acquiring unit configured to acquire a first radiation image, which is photographed by irradiating an object with radiation under a state in which a subject is not in an imaging range, and a second radiation image, which is photographed by radiating radiation under a state in which the subject and the object are in the imaging range; and
an image processing unit configured to perform correction for deleting image information on the object from the second radiation image by using the first radiation image.

2. A radiation imaging apparatus according to claim 1, wherein the image processing unit is configured to adjust a pixel value of the first radiation image to correct the second radiation image.

3. A radiation imaging apparatus according to claim 1,
wherein the first radiation image is acquired by radiating the radiation from a radiation generation unit, which is rotating about the object, at an arbitrary angle, and
wherein the second radiation image is acquired by radiating the radiation from the radiation generation unit, which is rotating about the subject and the object, at the arbitrary angle.

4. A radiation imaging apparatus according to claim 1, further comprising an offset image acquiring unit configured to acquire an offset image without radiating radiation in the imaging range,
wherein the image processing unit is configured to perform offset calibration on the second radiation image by using the offset image.

5. A radiation imaging apparatus according to claim 1,
wherein the image acquiring unit is configured to acquire a plurality of first radiation images of a plurality of objects, and
wherein the image processing unit is configured to select the first radiation image corresponding to the object in the second radiation image from among the plurality of first radiation images, and to correct the second radiation image by using the selected first radiation image.

6. A radiation imaging apparatus according to claim 1, further comprising a display unit configured to switch and display the second radiation image and the second radiation image corrected by using the first radiation image.

7. A radiation imaging apparatus according to claim 1, further comprising a display unit configured to generate a combined image by combining the object in the first radiation image with the second radiation image corrected using the first radiation image, and to switch and display the combined image and the second radiation image corrected by using the first radiation image.

8. A radiation imaging system, comprising:
a radiation generation unit, which is rotatable about a subject, and is configured to generate radiation;
a radiation detection unit, which is arranged facing the radiation generation unit, is rotatable together with the radiation generation unit, and is configured to detect the radiation;
an image acquiring unit configured to acquire a first radiation image, which is photographed by irradiating an object with radiation under a state in which the subject is not in an imaging range, and a second radiation image, which is photographed by radiating radiation under a state in which the subject and the object are in the imaging range; and
an image processing unit configured to perform correction for deleting image information on the object from the second radiation image by using the first radiation image.

9. A radiation imaging method, comprising:
acquiring a first radiation image, which is photographed by irradiating an object with radiation under a state in which a subject is not in an imaging range, and a second radiation image, which is photographed by radiating radiation under a state in which the subject and the object are in the imaging range; and
performing correction for deleting image information on the object from the second radiation image by using the first radiation image.

10. A non-transitory computer-readable medium having stored thereon a program to be executed by a processor to cause the processor to execute each step of the radiation imaging method of claim 9.
